(19) United States
(12) Patent Application Publication
(10) Pub. No.: US 2007/0198046 A1
(43) Pub. Date: Aug. 23, 2007

(54) Surgical visualization tool

(75) Inventors: Edwin J. Hlavka, Minneapolis, MN (US); Gregory G. Brucker, Minneapolis, MN (US); Steven D. Savage, Paynesville, MN (US); Adam L. Berman, Edina, MN (US)

Correspondence Address: MERCHANT & GOULD PC, P.O. BOX 2903, MINNEAPOLIS, MN, 55402-0903, US

(73) Assignee: MedicalCV, Inc.

(21) Appl. No.: 11/356,581

(22) Filed: Feb. 17, 2006

(51) Int. Cl. A61M 29/00

(52) U.S. Cl. 606/192

(57) ABSTRACT

The tool for enhancing visualization during surgery includes a shaft having a balloon member at a distal end. A fluid conduit through the shaft permits passing and inflation fluid into the balloon to selectively inflate the balloon. The balloon is formed of a material transparent to a wavelength of interest. A waveguide extends within the shaft into the interior of the balloon to visualize anatomical features external to the balloon.

### I. BACKGROUND OF THE INVENTION

1. Field of the Invention

This invention pertains to a surgical tool. More particularly, this invention pertains to a surgical tool to provide enhanced visualization during a surgical procedure.

2. Description of the Prior Art

A number of different methods have been suggested for creating lesions around pulmonary veins for the purpose of treating atrial fibrillation. Examples of these can be found in U.S. Pat. Nos. 6,474,340; 6,805,129; 6,161,543; 6,314,962; 6,314,963; 6,474,240 and 6,949,095. Also, such procedures and related tools are described in U.S. Patent Application Publication Nos. US 2003/0029462; US 2004/0260278; US 2004/0054263; US 2004/147912; US 2004/0102771 and US 2002/0087151.

The foregoing patent applications and patents teach placing an ablation element on an epicardial surface of the heart. Preferably, a complete lesion is formed surrounding pulmonary veins to isolate the pulmonary veins during a so-called MAZE procedure. Ablation elements come in a wide variety of forms and can include ultrasound, radio frequency ablation, laser ablation as well as diffused light ablation.

While MAZE procedures can be performed during so-called open chest procedures, atrial fibrillation treatments are most preferably performed through minimally invasive or laparoscopic procedures. In such procedures, small incisions are formed through the patient's chest in between the ribs to provide access for several ports to pass visualization instruments and surgical tools.

Minimally invasive cardiac procedures present challenging surgical obstacles requiring novel techniques to safely accomplish a procedure in a timely manner. In part, such challenges include limited visibility during such procedures which may frustrate a surgeon's identification of important anatomical landmarks.

Numerous tools have been suggested to enhance visualization within a laparoscopic procedure. For example, U.S. Pat. No. 6,277,136 to Bonutti dated Aug. 21, 2001 teaches a balloon to develop an anatomical space through which a scope can be passed as illustrated in FIG. 21 of the '136 patent. Other such tools include U.S. Pat. No. 6,764,497 to Fogarty et al., dated Jul. 20, 2004 and U.S. Pat. No. 6,860,892 to Tanaka et al., dated Mar. 1, 2005. It is an object of the present invention to provide an improved tool for creating a volume and to provide enhanced visualization of anatomical features within the volume.

### II. SUMMARY OF THE INVENTION

According to a preferred embodiment of the present invention an apparatus is disclosed for enhancing visualization during surgery. The apparatus includes a shaft having a proximal end and a distal end. The proximal end has a handle for manipulation of the shaft. A balloon member is provided at a distal end of the shaft. A fluid conduit through the shaft is provided for passing an inflation fluid from the proximal end into the balloon to selectively inflate the balloon. The balloon is formed of a material transparent to a wavelength of interest such as the visible spectrum. A waveguide extends through the shaft from the proximal end towards the distal end. A distal end of the waveguide is positioned within the balloon to visualize anatomical features external to the balloon.

### III. BRIEF DESCRIPTION OF THE DRAWINGS

FIG. 1 is a side elevation view, shown partially in section, of a visualization tool according to the present invention;

FIG. 2 is a side elevation view, shown partially in section, of the distal end of the tool of FIG. 1 shown in an enlarged format;

FIG. 3 is the view of the tool portion of FIG. 2 inserted within an anatomical space prior to enhancing the space for visualization;

FIG. 4 is the view of FIG. 3 following inflation of a balloon at a distal end of the tool to enlarge an anatomical space for enhanced visualization;

FIG. 5 is a side elevation view of an alternative embodiment of an enhanced visualization tool with a distal end shown in a first configuration;

FIG. 6 is the view of FIG. 5 with the distal end shown in a second configuration;

FIG. 7 is a view of the distal end of the tool of FIG. 5 shown in an anatomical space prior to inflation of a balloon; and

FIG. 8 is the view of FIG. 7 subsequent to inflation of a balloon at the distal end of the tool.

### IV. DESCRIPTION OF THE PREFERRED EMBODIMENT

With reference now to the various drawing figures in which identical elements are numbered identically throughout, a description of a preferred embodiment to the present invention will now be provided.

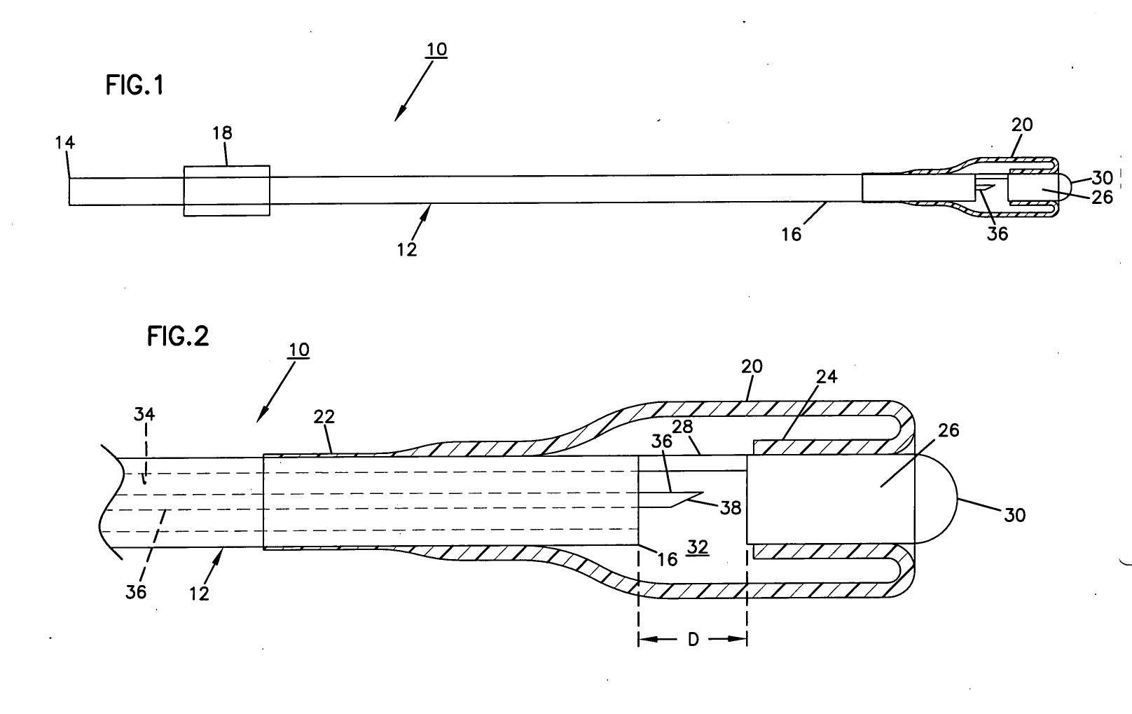

FIG. 1 illustrates the tool of the present invention in a first preferred embodiment. The tool includes a shaft 12 having a proximal end 14 and a distal end 16. Near the proximal end 14, a handle 18 is provided for a surgeon to grasp when manipulating the tool 10.

As shown best in FIG. 2, a balloon 20 is provided at the distal end 16. The balloon 20 is an elastomeric material which, in a preferred embodiment, is a clear transparent material which is transparent to the visible spectrum. However, the material of the balloon 20 could be selected to be any elastic material which is transparent to any other wavelength of interest. Opposing surfaces of the balloon 20, hub 26 and shaft 16 define an enclosed volume within the interior of the balloon 20.

A proximal end 22 of the balloon is secured to the shaft 12 to any suitable means such as adhesives, ultrasonic welding or the like. A distal end 24 of the balloon 20 is similarly secured to a hub 26. The hub 26 is coaxially aligned with the shaft 12 and spaced therefrom by a distance D. The spacing of the hub 26 from the shaft 12 is maintained by a rigid rod 28 which rigidly connects the rigid shaft 12 to the rigid hub 26.

In a preferred embodiment, the hub 26 terminates at a blunt rounded end 30. The blunt rounded end 30 permits the distal end 30 to be passed and urged by the surgeon through a dissection plane without presenting significant risk of trauma to opposing tissue. Alternatively, the end 30 could be any dissection tool (such as dissection jaws well known in the art) which can be manipulated by the handle 18 through mechanisms well known in the art.

The shaft 12 has a lumen 34 for passing an inflation fluid (such as a gas or a liquid, for example, saline) from the proximal end 14 of the shaft into the internal volume 32 of the balloon 20. FIG. 2 shows the balloon in a deflated state. Admission of an inflation fluid into the volume 32 inflates the balloon 20 to an expanded shape shown in FIG. 4.

A waveguide 36 extends through the lumen 34 and terminates at a distal end 38 positioned within the interior volume 32. The waveguide 36 is a conventional scope well-known in laparoscopic procedures and may be physically secured to the shaft 12 or, more preferably, advanced through the lumen 34 when desired.

In the embodiments of FIGS. 1-4, the waveguide 36 has an angled distal end 38 to provide a field of view F (shown in FIG. 4) which extends radially from the longitudinal axis of the shaft 12. Preferably, the scope 36 may be axially moved within the shaft 12 as well as rotatably moved within the shaft 12 relative to the longitudinal axis of the shaft 12.

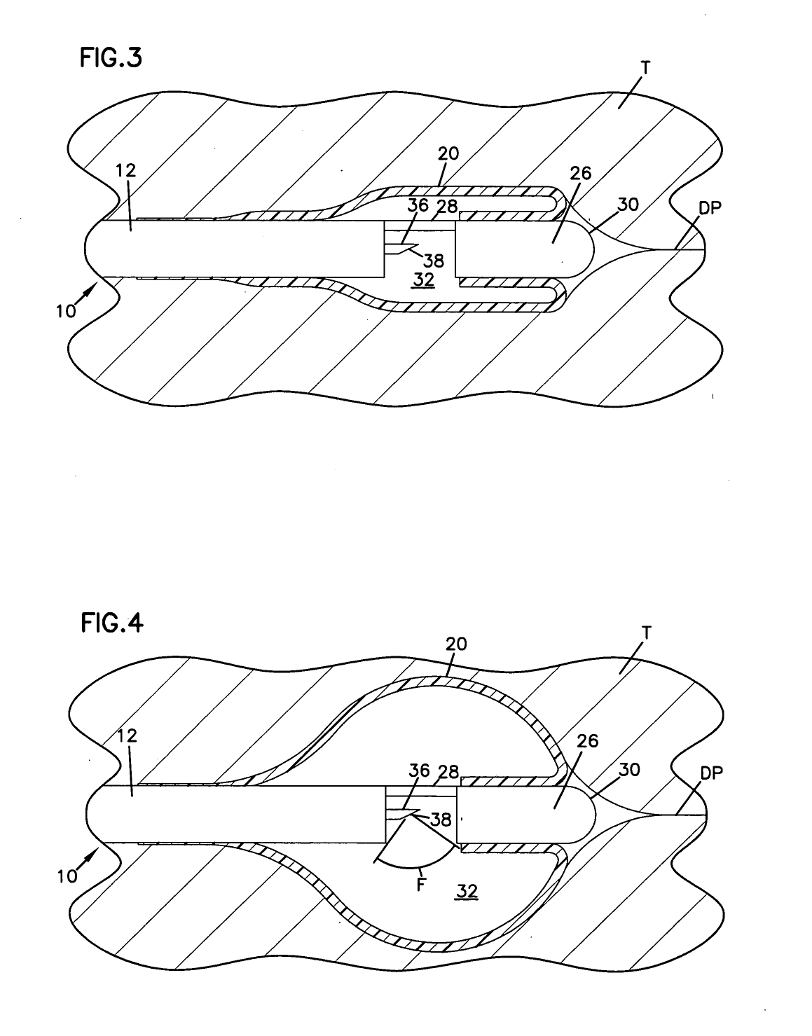

FIG. 3 illustrates advancement of the tool 10 through a tissue T. The blunt end 30 gently separates the tissue along a dissection plane DP which may occur within the tissue T. At any desired location, the surgeon may inflate the balloon to the inflated state of FIG. 4 to enhance the field of view F of the scope 36. Since the balloon 20 is transparent to the visible spectrum, the surgeon can use the scope 36 to inspect anatomical features through the walls of the balloon 20. The balloon 20 prevents anatomical fluids or other anatomical debris from obstructing the scope 36.

FIGS. 5-8 illustrate an alternative embodiment of a tool 40. The tool 40 has a longitudinal rigid shaft 42 with a handle 44 near a proximal end 46. The proximal end 46 is provided with the plurality of couplings 48 well known in the art for connecting an interior lumen (not separately shown) of the shaft 42 with sources of an inflation fluid as well as for advancing a scope through the lumen of the shaft 42 as will be described.

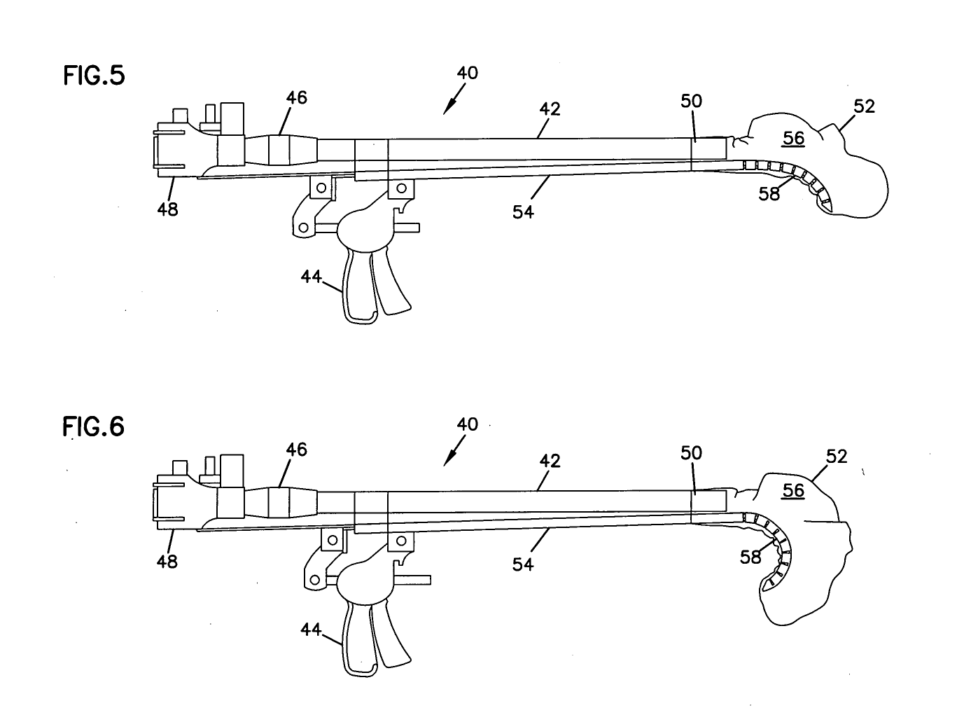

The distal end 50 of the shaft 42 carries a balloon 52 which in a preferred embodiment is a transparent non-distendable material. An articulating rod 54 is coupled to the shaft 42 and extends into the interior 56 of the balloon 52. Through ratcheting mechanisms well known in the art, the handle 44 is coupled to the rod 54 such that a distal end 58 of the rod (positioned within the balloon volume 56) may be curved from a straight configuration to a slightly curved configuration shown in FIG. 5 and into a fully curved configuration shown in FIG. 6.

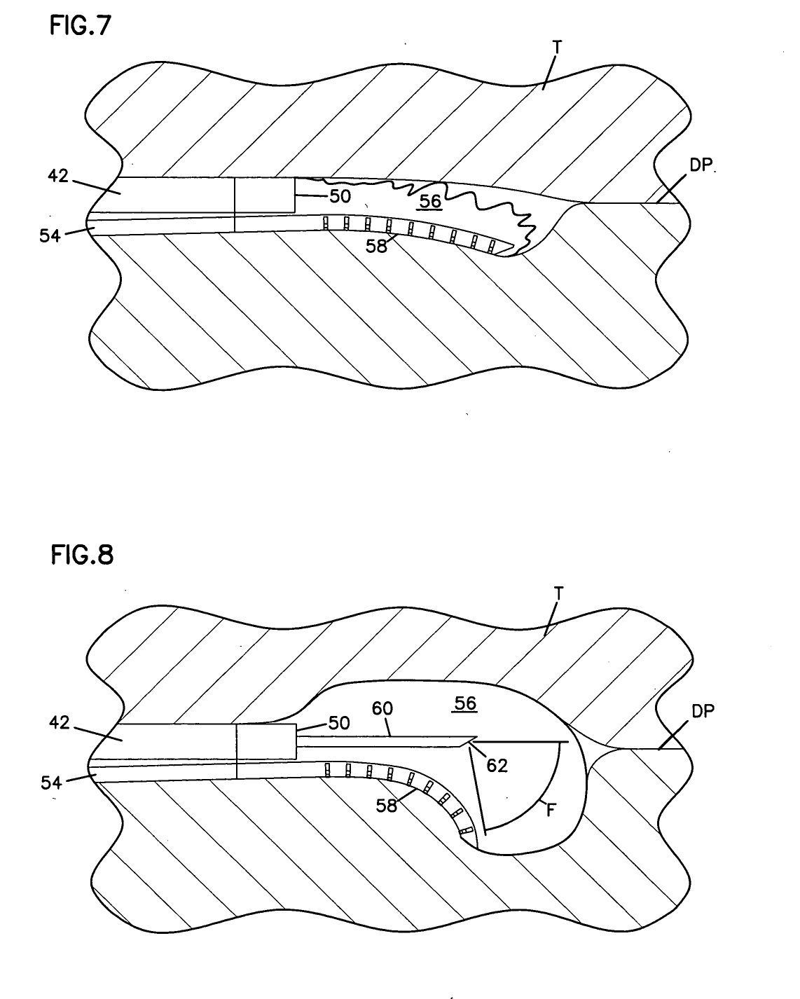

FIG. 7 illustrates advancement of the balloon and distal end 58 of the rod into a tissue T along a dissection plane DP. In FIG. 7, the balloon 52 is shown in a deflated state and the distal end 58 of the rod 54 is shown in an extended substantially straight configuration. At any desired location, a surgeon may admit an inflation fluid through the shaft into the interior 56 of the lumen 52. Such inflation of the balloon 52 enhances the volume of the anatomical space. Further, the volume can be additionally enhanced by manipulating the handle 44 to curve the rod distal end 58 to a curved state shown in FIG. 8.

The surgeon can then advance a scope 60 through the shaft 42 into the interior 56 of the balloon 52. As in the previous embodiment, the scope 60 preferably has a beveled end 62 to provide a field of view F which is, at least in part, radial to the longitudinal axis of the shaft 42. In addition to being axially movable within the shaft 42, the scope 60 may also be rotated about its longitudinal axis to provide a 360° view of the anatomical structure opposing the balloon 52.

It has been shown how the objects of the invention have been attained in the preferred embodiment. Modifications and equivalents of the disclosed concepts such as those which readily occur to one of skill in the art are intended to be included within the scope of the claims which are appended hereto.

We claim:

1. An apparatus for enhancing visualization during surgery, said apparatus comprising:
a shaft having a proximal end and a distal end with said proximal end having a handle for manipulation of said shaft;
a balloon member at said distal end;
a fluid conduit through said shaft for passing an inflation fluid from said proximal end into said balloon to selectively inflate said balloon;
said balloon formed of a material transparent to a wavelength of interest a waveguide extending within said shaft from said proximal end toward said distal end with a distal end of said waveguide positional within said balloon to visualize at said wavelength external to said balloon.

2. An apparatus according to claim 1 wherein said wavelength includes at least a portion of a visual spectrum.

3. An apparatus according to claim 1 wherein said distal end of said waveguide is axially movable within said balloon.

4. An apparatus according to claim 1 wherein said distal end of said waveguide is rotationally movable within said balloon.

5. An apparatus according to claim 1 wherein said balloon terminates at a blunt distal end.

6. An apparatus according to claim 1 wherein said balloon terminates at a dissection member.

7. An apparatus according to claim 1 wherein said balloon is bendable about a longitudinal axis extending axially away from said shaft.
